(19) United States
(12) Patent Application Publication
(10) Pub. No.: US 2006/0269919 A1
(43) Pub. Date: Nov. 30, 2006

(54) Method of diagnosis of obesity

(76) Inventors: Philippe Boutin, Tourcoing (FR); Severine Dubois, Vecquemont (FR); Christian Dina, Paris (FR); Philippe Froguel, Bagnolet (FR)

Correspondence Address: SUGHRUE MION, PLLC, 2100 PENNSYLVANIA AVENUE, N.W., SUITE 800, WASHINGTON, DC, 20037, US

(21) Appl. No.: 10/538,922

(22) PCT Filed: Oct. 31, 2003
(86) PCT No.: PCT/IB03/05282

§ 371 Date: Jun. 7, 2006

(30) Foreign Application Priority Data
Dec. 13, 2002 (EP) 022930853

(51) Int. Cl. C12Q 1/68; A61K 31/195

(52) U.S. Cl. 435/6; 514/561

(57) ABSTRACT

The invention relates to new methods of diagnosis and therapy of obesity, in particular morbid obesity, based on the identification of polymorphisms in the 5′ region of the gad2 gene.

The invention is in the field of human genetics and relates to new methods of diagnosis and therapy of obesity, in particular morbid obesity, based on the identification of polymorphisms in the 5′ region of the gad2 gene.

Morbid obesity is a serious disease process, in which the accumulation of fatty tissue on the body becomes excessive, interferes with, or injures the other bodily organs, and, creates (or predictably will create) serious and life-threatening health problems, which are called co-morbidities.

It is said that people are morbidly obese when their Body Mass Index (BMI=height (cm)/(mass (kg))²) is equal to or over 40.

Numerous scientific studies have established that there is a genetic predisposition to morbid obesity.

A region of the chromosome 10 has been involved in obesity (Hager et al, Nature Genetics 1998; 20:304-38.), which has been recently confirmed in a cohort of German young obese subjects (Hinney et al. 2000, Journal of Clinical Endocrinology and Metabolism 2000; 85 (8): 2962-5), as well as in White Caucasians and in African Americans (Price et al. 2001, Diabetologia 1999, 42: 555-9), and in the old order Amish (Hsuch et al. 2001, The Journal of Clinical Endocrinology and Metabolism 2001; 86(3): 1199-1205).

Three new single nucleotide polymorphisms (SNPs) have been identified in the frame of the invention, located in this region, and more particularly in the 5′ region of the gad2 gene. These SNPs show positive association with obesity in morbidly obese subjects, while other SNPS in this region do not show this association.

In the frame of the present invention, by gad2 gene is meant to indicate a gene whose coding sequence is represented by SEQ ID No 1.

The 5′ flanking region of gad2 gene is meant to indicate the region comprising nucleotides 1-2379 of SEQ ID No 2, and especially the region consisting in nucleotides 1-2379 of SEQ ID No 2. Nucleotides 2380-2382 of SEQ ID No 2 represent the start codon of the gad2 gene (ATG).

Thus, in a first embodiment, the invention relates to a method for diagnosing a predisposition for obesity, and in particular morbid obesity, in a human subject which comprises determining whether there is a germline alteration in the sequence of the 5′ flanking region of the gad2 gene, wherein said alteration is the presence of at least one of the following mutations: −243 A>G at nucleotide 2137 of SEQ ID No 2, −1.6 kb G>A at nucleotide 780 of SEQ ID No 2, −2004 A>T at nucleotide 376 of SEQ ID No 2, said alteration being indicative of a predisposition to obesity.

The 5′ flanking region of the gad2 gene is represented by nucleotides 1-2379 of SEQ ID No 2, and the SNPs according to the invention are located at nucleotides 376 (presence of a T in predisposed patients, and a A in non-predisposed patients), 780 (presence of a A in predisposed patients, and a G in non-predisposed patients), and 2137 (presence of a G in predisposed patients, and a A in non-predisposed patients) of SEQ ID No 2.

The invention relates more generally to the study of the 5′ promoter region of the gad2 gene. Indeed, the inventors have demonstrated that the presence of the specific SNPs indicated in the present application leads to increased binding of nuclear factors to the 5′ region of the gad2 gene, thus leading to an increase in transcription.

It is thus credible to speculate that other modifications in the promoter region of the gad2 gene, leading to a higher expression of the GAD2 protein will also lead to predisposition to obesity, due to increase in the GABA pool. Due to the orexigenic effect of GABA, this may alter feeding behavior.

Thus, in one embodiment, the invention relates to a method for diagnosing a predisposition for obesity in a human subject, wherein the level of an expression product of the gad2 gene in said sample is investigated.

The expression products according to the invention are meant to comprise RNA or protein, or what could be called “secondary” expression product, such as GABA. The latest is indeed not really an expression product of the gad2 gene, but increase in the production of GAD2 protein leads to increase in the GABA concentration.

Alteration of mRNA expression can be detected by any techniques known in the art. These include Northern blot analysis, PCR amplification and RNase protection

In one embodiment, mRNA of the sample is contacted with a gad2 gene probe under conditions suitable for hybridization of said probe to a RNA corresponding to said gad2 gene and hybridization of said probe is determined, and the level of signal after hybridization is compared with a standard (reference) signal (which is obtained from either a non-obese patient, or an obese patient).

The hybridization complex emits a signal, which may be due to the labeling of the probe, or of the mRNA. The different labels that may be used are well known to the person skilled in the art, and one can cite ³²P, ³³P, ³⁵S, ³H or ¹²⁵I. Non radioactive labels may be selected from ligants as biotin, avidin, streptavidin, dioxygenin, haptens, dyes, luminescent agents like radioluminescent, chemoluminescent, bioluminescent, fluorescent or phosphorescent agents.

In order to identify the SNPs mentioned in the present application, it is possible to contact a gad2 gene 5′ region probe with genomic DNA isolated from said sample under conditions suitable for hybridization of said probe to said gene and hybridization of said probe is determined.

Said gad2 gene 5′ region probe is either a “wild-type” DNA, (i.e. the searched SNP is not present on the probe, and hybridization occurs when no SNP according to the invention is present on the DNA in the sample) or a “mutant” one (i.e. carrying the searched SNP, and hybridization only occurs if the SNP is present on the DNA in the sample).

The person skilled in the art knows the techniques to determine the allele specific probes to use in this embodiment, their lengths, or the hybridization conditions. High stringency conditions are preferable in order to prevent false positives, for example under high stringency conditions of 0.2×SSC and 0.1% SDS at 55-65° C., or under conditions as described below.

The stringent hybridization conditions may be defined as described in Sambrook et al. ((1989) Molecular cloning: a laboratory manual. 2^{nd} Ed. Cold Spring Harbor Lab., Cold Spring Harbor, N.Y.), with the following conditions: 5× or 6×SCC, 50-65° C. Highly stringent conditions that can also be used for hybridization are defined with the following conditions: 6×SSC, 60-65° C.

Hybridization ADN-ADN or ADN-ARN may be performed in two steps: (1) prehybridization at 42° C. pendant 3 h in phosphate buffer (20 mM, pH 7.5) containing 5 or 6×SSC (1×SSC corresponding to a solution 0.15 M NaCl+0.015 M sodium citrate), 50% formamide, 7% sodium dodecyl sulfate (SDS), 10× Denhardt's, 5% dextran sulfate et 1% salmon sperm DNA; (2) hybridization during up to 20 at a temperature of 50-65° C., more preferably 60-65° C. followed by different washes (about 20 minutes at in 2×SSC+2% SDS, then 0.1×SSC+0.1% SDS). The last wash is performed in 0.2×SSC+0.1% SDS for about 30 minutes at about 50-65° C., and/or in 0.1×SSC+0.1% SDS at the same temperature. These high stringency hybridization conditions may be adapted by a person skilled in the art. Indeed, the person skilled in the art is able to determine the best stringency conditions by varying the concentrations in SSC and SDS and the temperature of hybridization and washings.

In another embodiment, said expression product is the polypeptide encoded by the gad2 gene in said sample. In particular, said polypeptide may be detected by immunoblotting or immunocytochemistry.

Antibodies specific for GAD2 can be used to detect increased GAD2 expression. Immunological assays can be done in any convenient formats known in the art. These include Western blots, immunohistochemical assays and ELISA assays.

Thus, antibodies that react with the the GAD2 polypeptide, as well as reactive fragments of such antibodies, are also encompassed within the scope of the present invention. The antibodies may be polyclonal, monoclonal, recombinant, chimeric, single-chain and/or bispecific. In preferred embodiment, the antibody or fragment thereof will either be of human origin, or will be “humanized”, i.e., prepared so as to prevent or minimize an immune reaction to the antibody when administered to a patient.

The antibody fragment may be any fragment that is reactive with the polypeptides of the present invention. the invention also encompasses the hybridomas generated by presenting the polypeptide according to the invention or a fragment thereof as an antigen to a selected mammal, followed by fusing cells (e.g., spleen cells) of the mammal with certain cancer cells to create immortalized cell lines by known techniques, such as the technique of Köhler et Milstein (1975 Nature 256, 495).

The antibodies according to the invention are, for example, chimeric antibodies, humanized antibodies, Fab ou F(ab′)₂ fragments. They may be immunoconjugates or labeled antibodies, for detection purposes.

In another embodiment, the invention is performed by determining whether there is an alteration in the germline sequence of the gad2 gene 5′ region in said sample by observing shifts in electrophoretic mobility of single-stranded DNA from said sample on denaturing or non-denaturing polyacrylamide gels. Said single-stranded nucleic acids may be obtained after amplification of the genomic DNA, using suitable primers, and denaturation (the gel and electrophoresis conditions are usually denaturing for such a purpose).

In another embodiment, the invention is performed by amplification of all or part of the gad2 gene 5′ region from said sample, and determination of the sequence of said amplified DNA, for example by chain termination extenstion.

In another embodiment, allele specific oligonucleotide primers are employed to determine whether a specific gad2 mutant allele is present in said sample, the amplification only occurring in this case. This method is well known in the art.

In another embodiment, all or part of the gad2 gene 5′ region from said sample is cloned to produce a cloned sequence and the sequence of said cloned sequence is determined. The cloning is performed in vectors known in the art, such as pUC or pBR vectors.

In general, the invention relates to a method for determining a predisposition to obesity, in particular morbid obesity, in a subject, from a sample of said subject, which comprises determining whether there is a mismatch between (1) the 5′ region of the gad2 gene genomic DNA isolated from said sample, and (2) a nucleic acid probe complementary to human wild-type 5′ region of the gad2 gene DNA, as represented by SEQ ID No 2, when molecules (1) and (2) are hybridized to each other to form a duplex, and said mismatch being due to the presence of at least one of the 3 identified SNPs, present et nucleotides 376, 780 or 2137 of SEQ ID No 2.

In another embodiment, the invention relates to a method wherein amplification of gad2 gene 5′ flanking region sequences in said sample is carried out and hybridization of the amplified sequences to one or more nucleic acid probes issued from the wild-type gad2 gene 5′ flanking region sequence (as represented in SEQ ID No 2) or a mutant gad2 gene 5′ flanking region sequence (in which at least one of the three SNPs located at nucleotides 376, 780 or 2137 of SEQ ID No 2 is present), is determined.

The invention also relates to a a method which comprises determining in situ hybridization of the gad2 gene 5′ flanking region in said sample with one or more nucleic acid probes issued from a wild-type or a mutant gad2 gene 5′ flanking region sequence.
In another embodiment, the invention is aimed at a primer or probe for detecting a predisposition for obesity selected from SEQ ID No 4 to 15.
It also encompasses a kit for detecting a predisposition for obesity comprising a set of primers or probes consisting of SEQ ID No 4, 5, 8, 9, 12 and 13 or a set of primers or probes consisting of SEQ ID No 6, 7, 10, 11, 14, and 15.
This kit may further comprise a primer or probe allowing detection of a protective haplotype consisting of 10 to 30 consecutive nucleotides of SEQ ID No 16 or 17 or of a sequence complementary thereof.

The invention opens up a new area in the treatment and/or prevention of obesity, in particular morbid obesity, especially for patient in families where genetic susceptibility is suspected.

Indeed, the presence of the SNPs in the 5′ region of the gad2 gene, and the data showing increased binding of nuclear factors to said region when the polymorphisms identified in the frame of the invention are present, make it likely and credible that presence of these polymorphisms increase expression of gad2, probably through increased transcription. Furthermore, increased activity of gad2 gene leads to increase of the GABA pool, of the orexigenic effect of GABA, which may alter feeding behavior and contribute to the development of morbid obesity for the carriers of the identified mutations.

Thus, the invention also relates to a method for screening potential obesity drugs which comprises: combining (i) a compound suspected of being a obesity drug, (ii) a GAD2 polypeptide and determining the amount of binding of the GAD2 polypeptide to said compound. Indeed, inhibitors of the GAD2 enzyme will interfere with the formation of GABA and can be considered as useful drugs for treating obesity, alone or in association with other treatments.

The invention also relates to a method for screening potential obesity therapeutics which comprises: combining (i) a GAD2 binding partner, (ii) a GAD2 polypeptide and (iii) a compound suspected of being a obesity therapeutic and determining the amount of binding of the GAD2 polypeptide to its binding partner.

In a particular embodiment, said binding partner is L-glutamic acid.

The invention also relates to a method for screening potential obesity therapeutics which comprises: combining (i) a gad2 gene binding partner, (ii) a gad2 gene and (iii) a compound suspected of being a obesity therapeutic and determining the amount of binding of the gad2 gene to its binding partner. In this embodiment, the term “gad2 gene” must be understood as including the 5′ flanking region in the gad2 locus, which comprises the polymorphisms of the invention, especially −243 A>G.

In particular, said gad2 gene binding partner is IK2 (Ikaros 2), the amino acid sequence of which is represented by SEQ ID No 3.

The methods for assessing the binding of a compound to a nucleic acid or a protein are well known in the art, and are preferably performed in vitro. A method to achieve such a goal may be to link the nucleic acid or the protein to a solid support on which the compound to test is flown, and to check the recovery of the compound after passage on the support. By adjusting parameters, it is also possible to determine the binding affinity. The compounds may also be found by other methods including FRET, SPA . . . when the compounds and the nucleic acid or protein are labeled. The assay may also be performed on the cells containing the nucleic acid of the invention, for example on a vector according to the invention, and/or expressing the protein according to the invention. This also gives the information of the capacity of the compound to go through the membrane and penetrate within cells. These cells can be bacterial cells, or mammalian cells.

The method of the invention also allows the screening, detection and/or identification of compounds able to inhibit the biological activity of the GAD2 protein, and n particular the increase of the amount of produced GABA.

The present invention thus allows the detection, identification and/or screening of compounds that may be useful for the treatment of diseases where V(D)J recombination and/or DNA repair is involved. Nevertheless, the compounds identified by the method according to the invention, in order to be used in a therapeutic treatment, may need to be optimized, in order to have a superior activity and/or a lesser toxicity.

Indeed, the development of new drugs is often performed on the following basis:
screening of compounds with the sought activity, on a relevant model, by an appropriate method,
selection of the compounds that have the required properties from the first screening test (here, modulation of GAD2 or GABA production),
determination of the structure (in particular the sequence (if possible the tertiary sequence) if they are peptides, proteins or nucleic acids, formula and backbone if they are chemical compounds) of the selected compounds,
optimization of the selected compounds, by modification of the structure (for example, by changing the stereochemical conformation (for example passage of the amino acids in a peptide from L to D), addition of substituants on the peptidic or chemical backbones, in particular by grafting groups or radicals on the backbone, modification of the peptides (se in particular Gante “Peptidomimetics”, in Angewandte Chemie-International Edition Engl. 1994, 33. 1699-1720),
testing and screening of the “optimized” compounds on appropriate models that are often models nearer to the studied pathology. At this stage, one would often use animal models, in particular rodents (rats or mice) or dogs or non-human primates, that are good models of obesity, and to look for the phenotypic changes in said models after administration of the compound.

The present invention also encompasses the compounds that have been optimized after following the steps or equivalent steps as described.

The invention also relates to a pharmaceutical composition comprising a pharmaceutically acceptable excipient and a compound identified by a method according to the invention, and to the use of a compound identified by a method according to the invention for the manufacture of a drug intended to treat and/or prevent obesity, especially morbid obesity.

The invention also relates to a method for the therapy of obesity, in particular morbid obesity, comprising administration of a compound or a pharmaceutical composition of the invention. In order to decrease the activity of the gad2 gene and protein, it is possible to use antisense molecules or SiRNA, in particular complementary to the mRNA of gad2, in a pharmaceutical composition for treating obesity.
The invention also relates to the use of a compound as defined above or a antisense molecules or SiRNA for the modulation of insulin secretion.

The invention relates to a method for the therapy of obesity, comprising administration of anti-gad2 antisense molecule or any means to decrease the amount of GAD2 protein. The person skilled in the art is aware of the means to design antisense molecules, and of the modifications that can be brought to the backbones of the molecules (phosphorothioates, methylphosphonates . . . ).

Antisense polynucleotide sequences are useful in preventing or diminishing the expression of the gad2 gene, as will be appreciated by those skilled in the art. For example, polynucleotide vectors containing all or a portion of the gad2 gene (or other sequences from the gad2 locus, especially the 5′ flanking region) may be placed under the control of a promoter in an antisense orientation and introduced into a cell. Expression of such an antisense construct within a cell will interfere with gad2 transcription and/or translation.

Alternatively, a “sense” strategy may be foreseen, where a nucleic acid oligonucleotide or probe comprising part of the 5′ region of the gad2 gene (especially comprising the −243 A>G variant at nucleotide 2137 of SEQ ID No 2) is introduced within the cells, in order to be competitor for binding of the nuclear factors activating transcription. The size of the fragment of the 5′ flanking region of the gad2 gene that can be used in the sense strategy is preferably about 50-150 bases.

The invention also relates to new animal models useful for studying obesity, where expression of the gad2 gene is increased. In particular, expression of this gene can be increased only conditionally, using promoters that are either site- or time-specific, or inducible promoters. It is thus possible to increase gad2 expression only in the pancreas of the animals according to the invention, or in the brain. The methods to obtain animals according to the invention are known by the persons skilled in the art, and are useful in particular for testing some drugs that can be identified according to the methods of the invention.

The insertion of a construct in the genome of an animal to obtain a transgenic animal may be performed by methods well known by the artisan in the art, and can be either random or targeted. In a few words, the person skilled in the art will construct a vector containing the sequence to insert within the genome, with an appropriate promoter, and a selection marker (for example the gene coding for the protein that gives resistance to neomycine), and may have it enter in the Embryonic Stem (ES) cells of an animal. The cells are then selected with the selection marker, and incorporated into an embryo, for example by microinjection into a blastocyst, that can be harvested by perfusing the uterus of pregnant females.

Reimplantation of the embryo and selection of the transformed animals, followed by potential back-crossing makes it possible to obtain such transgenic animal. To obtain a “cleaner” animal, the selection marker gene may be excised by use of a site-specific recombinase, if flanked by the correct sequences.

The invention thus relates to a transgenic non-human mammal having integrated into its genome the nucleic acid sequence of the gad2 gene or the coding sequence thereof, operatively linked to regulatory elements, wherein expression of said coding sequence increases the level of the GAD2 protein and/or the GABA pool in said mammal relative to a non-transgenic mammal of the same species.

The gad2 sequence foreseen for the preceding embodiment may be a human gad2 gene sequence introduced within the genome of the animal of the invention, or the endogenous gad2 gene sequence the promoter of which has been modified in order to induce overexpression. For example, the gad2 coding sequence for mouse is knoan and may be found in GenBank under number NM_{—}008078. Other endogenous gad2 genes in other animals may be identified using the homologies between sequences.

Furthermore, the invention also relates to a transgenic non-human mammal whose genome comprises a disruption of the endogenous gad2 gene, as an animal model for testing links bewteen GAD2 and obesity. In particular, said disruption comprises the insertion of a selectable marker sequence, and said disruption results in said non-human mammal exhibiting a defect in GABA level as compared to a wild-type non-human mammal.

In particular, said disruption is a homozygous disruption, and said homozygous disruption results in a null mutation of the endogenous gene encoding GAD2.

U.S. Pat. No. 6,087,555 describes one way of obtaining a knock-out mouse, and the general teaching of this patent is incorporated herein by reference (column 5, line 54 to column 10 line 13). In this patent, it is described an OPG knock-out mouse, but the same method applies to a GAD2 knock-out mouse. The person skilled in the art will also find information in Hogan et al. (Manipulating the Mouse Embryo: a Laboratory Manual, Cold Spring Harbor Laboratory Press, Cold Spring Harbor, N.Y.; 1986).

The mammal of the invention is preferably a rodent, especially a rat or a mouse.

### DESCRIPTION OF THE FIGURES

FIG. 1: schematic representation of the gad2 gene, and of the frequency of the SNPs identified in the frame of the invention.

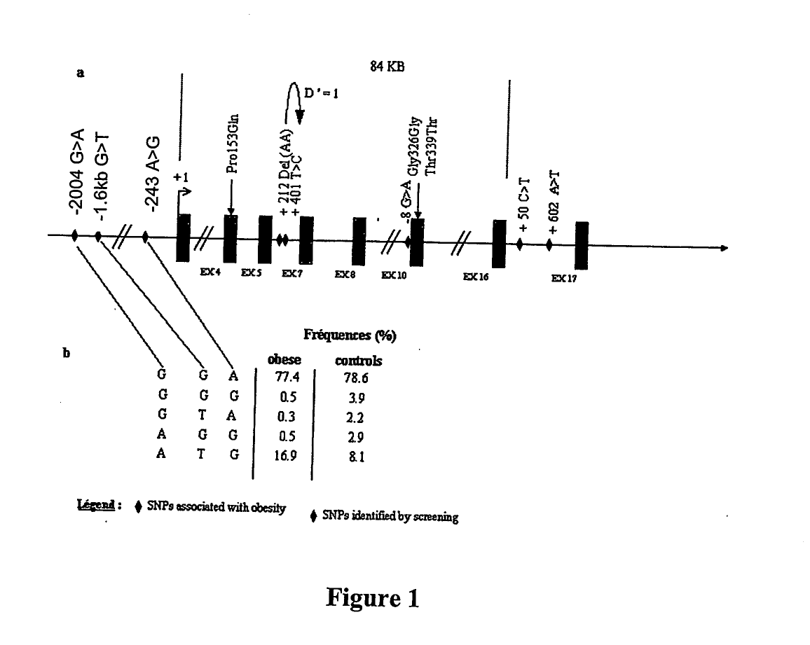

FIG. 2: gel shift assay, testing the binding of nuclear protein in the 5′ region of the gad2 gene, depending of the nature of the nucleotide at position −243. mt=mutant (G); wt=wild type (A).

FIG. 3: Effect of −243A>G gad variant on transcriptional activity in βTC3 cells.

Relative Luciferase Units are expressed as means±S.D.

### EXAMPLES

### Example 1

### Association Between Obesity and SNPs

The Gad2 gene, encodes an isoform of glutamic acid decarboxylase (GAD65), that catalyses the formation of gamma aminobutyric acid (GABA) from L glutamic acid, and is expressed in both brain and pancreatic islets cells. GABA is one of the most powerful inhibitory neurotransmitters of the central nervous system (CNS). GABA modulate the gastrin's and somatostatin's secretion and may stimulate the glucose intake in several tissues (Erdo and Wolff, 1990 J Neurochem.; 54(2):363-72.).

Three SNPs (−243 A>G variant (5′UTR1); -1.6 kb G>A (SNP 668); -2004 A>T (SNP 669)), located in the 5′ flanking region of the gad2 gene revealed positive association with obesity in morbidly obese subjects.

The DNA fragments bearing these polymorphisms can be amplified with the following sets of primers,

Polymorphisms of the gad2 gene were genotyped by direct sequencing or by the LightCycler™ technology (Roche, Manheim, Germany). The LightCycler assay is based on hybridization probes labelled with fluorescent dyes that allow fluorescence resonance energy transfer (Blomeke et al., 1999, Anal Biochem, 275, 93-7). SNP genotyping was carried out using melting curves analysis. PCR are performed in 9700 apparatus (Applied Biosytems, Foster city, USA) before analysis in lightCycler. Sequences of primers used for PCR and for the LightCycler assays are described in the following table.

| | |
|---|---|
| SNPs | PCR primers |

| | | |
|---|---|---|
| −243 A>G | F: 5′cctcaaatgctctggggctc3′ | SEQ ID No 4 |
| | R: 5′ggtgtcacgcaggaacagaa3′ | SEQ ID No 5 |
| −1600 G>A | F 5′ctgaggcgtattaggag | SEQ ID No 8 |
| | R: 5′ctcctaatacgcctcag | SEQ ID No 9 |
| −2004 A>T | F: 5′tgttttcaaccactcatccat | SEQ ID No 12 |
| | R; 5′agggacagttatgctcg | SEQ ID No 13 |

| | |
|---|---|
| SNPs | Primers for Light cycler assay |

| | | |
|---|---|---|
| −243 | Red640-gtctcttttaaagctccccggct | SEQ ID No 6 |
| A>G | | |
| | cgggctccgaggacccttaggtagtccc-F | SEQ ID No 7 |
| −1600 | Red640-ggaaagcagccgcctc | SEQ ID No 10 |
| G>A | | |
| | tggaaatgacaggcgctctggccaggcgcg- | SEQ ID No 11 |
| | F | |
| −2004 | Red640-acagcctggtacagactt | SEQ ID No 14 |
| A>T | | |
| | tgagttttcgaccacccgggctc-F | SEQ ID No 15 |

(F represents the fluorescein tag).

PCR amplifications were performed in a final reaction volume of 20 □l PCR buffer, containing 50 ng human genomic DNA, 10 pmol of each primer, 2,5 mmol/l MgCl2, 2,5 mmol/I of dNTP, and 0,2 U of Taq Gold polymerase. The thermal cycling conditions for the PCR product were: 1 cycle 95° C. 12 min; 35 cycles, 95° C. 15 sec, 55° C. 15 sec, 72° C. 30 sec; 1 cycle 72° C. 10 min; 1 cycle 15° C. 15 min.

In addition, in order to study the restrained eating and “latent obesity”, cognitive restraint of eating, disinhibition and hunger were analyzed according the “Three factor eating questionnaire or TFEQ” (Sturkard A J and Messick S, 1985 J Psychosom Res; 29(1):71-83.).

Association studies were performed in 369 morbidly unrelated obese patients (mean BMI: 47.3±7.4 kg/m2; mean age: 46±12 years; women/men, 294/75).

A set of 381 unrelated non obese non diabetic subjects (mean BMI: 22.8±2.44 kg/m²; mean age: 58.3±14.1 years; women/men, 228/152) was used as a control group. Table 1 shows genotype distribution and allele frequencies of the 3 SNPs (−243 A>G variant (5′UTR1); -1.6 kb G>A (SNP 668); -2004 A>T (SNP 669)).

TABLE 1
Genotype distribution and allele frequencies of 242 A > G,
1.6 kb G > A and 1.7 kb A > T SNPs

| | | Morbidly obeses | | | | |
|---|---|---|---|---|---|---|
| | | BMI > 40 | | Controls | | |
| | | num- | | num- | | |
| | | ber | | ber | | |
| | | of | | of | | |
| | Geno- | geno- | Allele | geno- | Allele | |
| SNP | type | types | Freq. | types | Freq. | p-value |

Codominant Model

| | | | | | | |
|---|---|---|---|---|---|---|
| 5′UTR: | AA | 233 | A: 79.9% | 270 | A: 84.5% | p = |
| −243 A > G | AG | 117 | G: 2o.1°io | 92 | 6: 15.5% | 0.05 |
| | GG | 15 | | 12 | | |
| SNP668: | GG | 229 | 6: 80.2% | 255 | 6: 84.8% | p = |
| −1.6 kb G > A | GA | 113 | A: 19.8% | g7 | A: 15.2% | 0.06 |
| | AA | 14 | | 10 | | |
| SNP669: | AA | 221 | A: 80.4% | 254 | A: 83.8% | p = |
| −2004 A > T | AT | 105 | T: 19.6% | g9 | T: 16.2% | 0.19 |
| | AA | 14 | | 13 | | |
| Dominant | | | | | | |
| Model | | | | | | |
| 5′UTR: | AA | 233 | A: 79.9% | 270 | A: 84.5% | p = |
| −243 A > G | AG/GG | 132 | 6: 20.1% | 104 | 6: 15.5% | 0.004 |
| SNP668: | GG | 229 | 6: 80.2% | 255 | 6: 84.8% | p = |
| −1.6 kb G > A | GA/AA | 127 | A: 19.8% | 97 | A: 15.2% | 0.02 |
| SNP669: | AA | 221 | A: 80.4% | 254 | A: 83.8% | p = |
| −2004 A > T | AT/AA | 119 | T: 19.6% | 102 | T: 16.2% | 0.07 |

Under codominant and dominant model, the −243 A>G and the −1.6 kb G>A SNP were associated with morbid obesity (respective p values of 0.004 and 0.02 under a dominant model). A trend toward association was observed for the −2004 A>T variant.

The relative risk of these 3 SNPs was estimated: O.R=1.55 (95% CI [1.14 2.12]) (UTR5I; −243); O.R=1.45 (95% CI [1.06 2]) (SNP668; −1.6 kb); O.R=1.34 (95% CI [0.97 1.84]) (SNP669; −2004).

In order to replicate these results, the 3 SNPs were genotyped in another set of 316 unrelated morbidly obese subjects. Similar result was obtained for the −243 A>G (under dominant model, p=0.009) and a trend toward was obtained for the −1.6 kb G>A SNP (under dominant model, p=0.06).

Analysis of variance for obesity related phenotypes was performed (BMI, leptin, percent fat mass, insulinemia . . . ) and significant association between the 3 SNPs and the three factors of eating behavior was found.

To test the human eating behavior, the subjects filled in the TFEQ (Three Factor Eating Questionnaire) questionnaire established by Stunkard et al. (op. cit.).

The analysis of variance for three stable factors measured by TFEQ: cognitive restraint of eating (Qres), disinhibition (Qdis) and hunger (Qhun) is show in table 2.

Table 2 shows the results obtained for the −243 A>G (5′UTR) variant. Similar results were obtained for the −1.6 kb G>A (SNP 668) and the −2004 A>T (SNP 669) variants.

TABLE 2
Analyses of variance with fond intake behavior parameters.

| | −243 A > G (5′UTRI) | | | |
|---|---|---|---|---|
| | AA | AG | GG | P |
| N | 233 | 117 | 15 | |
| Age | 46.02 ± 0.78 | 47.82 ± 1.1 | 39.9 ± 3.1 | 0.04 |
| Age-Pmax | 43.5 ± 0.79 | 44.3 ± 1.1 | 35.76 ± 3.1 | 0.03 |
| Qres | 9.53 ± 0.31 | 9 ± 0.45 | 6.53 ± 1.28 | 0.06 |
| Qdis | 8.38 ± 0.24 | 8.71 ± 0.34 | 10.9 ± 0.96 | 0.03 |
| Qhun | 5.29 ± 0.24 | 6.49 ± 0.35 | 7.95 ± 0.95 | 0.001 |

Using a codominant model, it was found that the GG carriers presented a lower restriction score than heterozygous and wild type subjects (pvalue=0.06). They also presented a higher disinhibition (pvalue=0.03) and a higher hunger scores (pvalue=0.001) than heterozygous and wild type subjects. Furthermore, it was observed that GG carriers are younger (pvalue=0.04) and seem to reach a maximal weight in early ages than AG and AA carriers (pvalue=0.03). These data suggest that alterations of feeding behavior by gad2 variants can contribute to the development of younger onset of morbid obesity.

### Example 2

### Functional Study of a Promoter Variant of gad 2 Gene

To search for potential binding sites, the sequence of the 5′ flanking region of the gad2 gene was submitted to the transfac server: http://transfac.gbf.de/cgi-bin/mat.

In silico analyses showed that the −243 A>G (located at nucleotide 2137 of SEQ ID No 2) and the −1.6 kb G>A (located at nucleotide 780 of SEQ ID No 2) were located near a predicted Ikaros 2 (IK2) response element site. IK2 is a transcription factor that is highly expressed in human lymphocytes, and its amino acid sequence is represented by SEQ ID No 3.

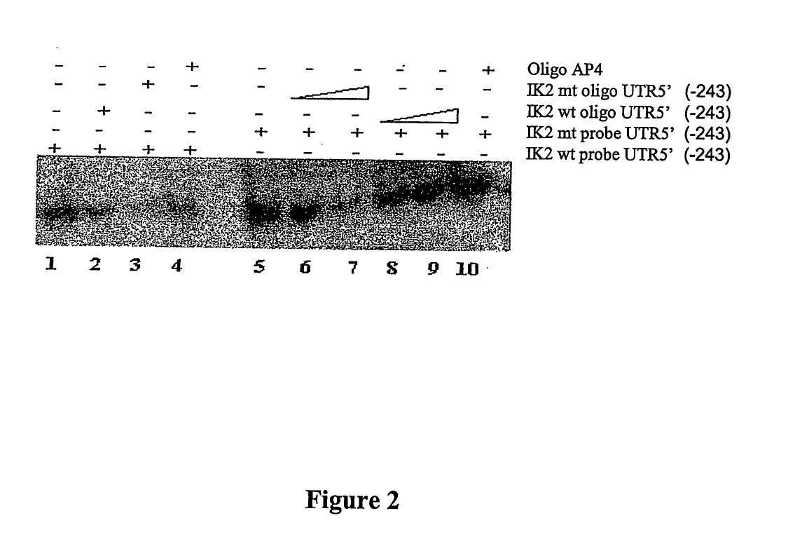

In order to test if the −243 A>G variant alters nuclear protein binding to the DNA, a gel shift assay using primers with sequences of the wild type and mutated site and a nuclear extract from MIN6 cells was performed (FIG. 2).

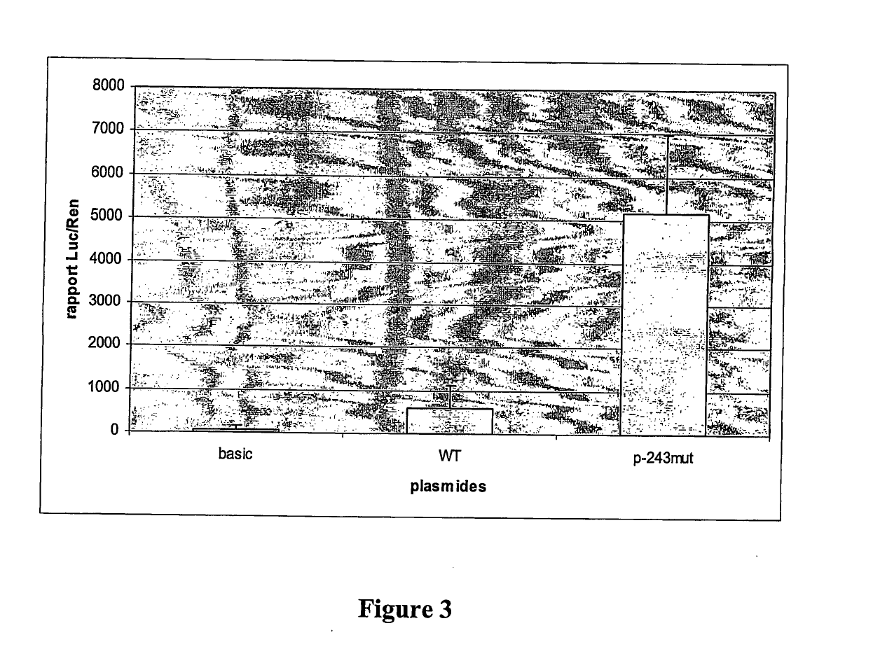

Results of the gel shift assay showed that there is in vitro binding of nuclear proteins to the IK2 responsive element site. Apparently, the nuclear proteins have a higher affinity to the response element G variant (lire 6 versus 1). Similar result were obtained with competition of the A and G allele (lire 2 vs 3; 6 vs 8; 7 vs 9). To investigate if the allelic variant at position −243 influences GAD2 transcriptional level, transient cotransfections using firefly luciferase reporter construct were performed to measure proximal promoter activity of wild type and variant promoters in βTC3 cells (murine insulinoma cell line). Renilla vector was used to normalize transfection efficiency. The transcriptional activity of −243A>G mutant construct was 8.61 times higher compared to the wild type construct (n=8, p<0.0001) (FIG. 3)

The observed increased transcriptional activity induced by the G allele of the −243 A>G is in concordance with the genetic results
the association of the −243 A>G variant with obesity (p=0.004, O.R=20=1.55 (95% CI [1.14-2.12])
The GG bearers have a lower food restriction score (p=0.06), a higher disinhibition score (p=0.03) and a higher hunger score parameter (p=0.001).

These results indicate that the binding of nuclear proteins (potentially the IK2 transcription factor), in presence of variant allele, was increased and may transactivate the Gad2 gene, and thus the GABA pool. Therefore, orexigenic effect of GABA might be increased and might alter feeding behaviour and contribute to the development of morbid obesity for G carriers of the −243 A>G variant.

The result may be generalized to the other SNPs, that are also close to IK2 response element.

The evidence presented in this application indicates involvement of the GABA pathway in obesity in human. This is an important step towards a better understanding of the molecular mechanisms leading to common forms of obesity. It seems clear that the GABAergic neurons are involved in the integration of signals modulating food intake. The identification of SNPs in the 5′ region of the gad2 gene and the demonstration that their presence modulates the expression of a key enzyme (GAD2), which in turn will modify the GABA pool opens new perspective for drugs against obesity.

Identification of a protective haplotype (for morbid obesity) including the most frequent alleles of SNP+61450 C>A, and +83897 T>A

### Example 3

### Identification of a Protective Haplotype (for Morbid Obesity) Including the most Frequent Alleles of SNP+61450 C>A, and +83897 T>A

Haplotype analyses of SNPs −243 A>G, +84 G>A, +61450 C>A and +83897 T>A were then performed by both HTR and PM+EH+ methods. Haplotypes including SNP +84 G>A with each of the three remaining SNPs did not improve association with morbid obesity and indeed association of SNP +84 G>A resulted from its LD with other three SNP (data not shown). Thus, SNP +84 G>A was excluded from further haplotype analyses. Haplotype structures including the three SNPs, −243 A>G, +61450 C>A, and +83897 T>A were then investigated in the 575 unrelated morbidly obese patients with familial history of obesity and in the 646 non obese subjects. Among the 8 possible haplotypes defined by SNPs −243, +61450 and +83897, only 6 haplotypes displayed a frequency>1% and their combined prevalence encompassed all but 2% of the haplotypes seen in the population (table 3).

TABLE 3
haplotype analysis of the obese status in 575 morbidly obese and 646
control subjects. Haplotypes covering three (−243, +61450, +83897)
and two (+61450, +83897) SNPs have been investigated.
Family-based association test for each haplotype. A positive Znorm
means that there is an excess of allele in affected offspring.

| | | | Case/control Test | | | | |
|---|---|---|---|---|---|---|---|
| Haplotypes | | | | Mor- | | Family-Based | |
| −243 | +61450 | +83897 | Non | bidly | | Association Test | |
| A > G | C > A | T > A | obese | obese | p-value | Znorm | p-value |
| 1_{(A)} | 1_{(C)} | 1_{(T)} | 70.6 | 64.3 | 0.003 | −1.92 | 0.05 |
| 2_{(G)} | 2_{(A)} | 2_{(A)} | 15 | 16 | 0.40 | 1.56 | 0.12 |
| 1_{(A)} | 2_{(A)} | 1_{(T)} | 11 | 12.8 | 0.023 | 1.15 | 0.25 |
| 2_{(G)} | 2_{(A)} | 1_{(T)} | 0.9 | 2.24 | 0.025 | | |
| 2_{(G)} | 1_{(C)} | 2_{(A)} | 0.09 | 1.3 | 0.003 | | |
| 1_{(G)} | 1_{(C)} | 2_{(A)} | 0.2 | 1 | 0.87 | | |
| | 1_{(C)} | 1_{(T)} | 71.2 | 65.3 | 0.0049 | −1.94 | 0.05 |
| | 2_{(A)} | 2_{(A)} | 16.6 | 17.1 | 0.59 | 1.73 | 0.08 |
| | 2_{(A)} | 1_{(T)} | 11.8 | 15.11 | 0.044 | 1.14 | 0.25 |
| | 1_{(C)} | 2_{(A)} | 0.4 | 2.4 | 0.0003 | | |

1 wild allele 2 variant allele;
The corresponding nucleotides are shown as subscripts in parentheses

Comparison of haplotype frequencies performed by haplotype trend regression (HTR) between cases and controls showed evidence for association with obesity with an empirical pvalue of 0.0004. This positive result was ascertained by a PM+EH+ analysis (p=0.0002). Frequencies of haplotypes bearing the wild type alleles (A-C-T) were significantly higher in non-obese compared to the morbidly obese group (table1). It is noteworthy that the risk G allele at SNP −243 A>G was present on several haplotypes with frequencies lower than 1% which cannot be taken into account for the haplotype analysis. SNPs −243, +61450 and +83897 were in strong linkage disequilibrium, the chi2 between these markers were ranging from 556 to 1265, thus p values were <0.00001.
In addition posterior probabilities of haplotype accuracy, as assayed by SNPHAP, were >0.98 for 98% of the subjects included in the study. For the remaining 2% of subjects, posterior probabilities were ranging from 0.88 to 0.98. Moreover on a background wild type CC for SNP+61450 C>A or TT for SNP +83897 T>A, association of SNP −243 A>G with morbid obesity remained significant (p=0.01 and p=0.03 respectively). The independent effect of −243 A>G was confirmed by haplotype analysis of SNPs +61450 C>A, and +83897 T>A that presented a significant association with morbid obesity, overall permutation showing an empirical pvalue<0.0001. Estimated frequencies of haplotype bearing the wild type alleles (C-T) remained higher in the non-obese compared to the morbidly obese group (p=0.0049). Thus the (C-T) haplotype displayed a “protective” effect against obesity (OR=0.81 95% CI [0.681-0.972])

### Example 4

### Linkage, Familial Association and Association with the Evidence of Linkage of GAD SNPs

Familial association and association with the evidence of linkage were investigated in the 188 nuclear families (612 individuals). First, as expected in a region of linkage, the SNPs −243 A>G, +61450 C>A and +83897 T>A showed significant linkage with a binary obese status, displaying MLS of respectively 2.54, 1.86 and 4.54 (data not shown. The SNPs of GAD certainly show linkage with obesity and that they are, therefore, worth investigating for family based association tests. Familial-Based Association Test (FBAT) was used to detect association in our established linkage context. In single SNP analyses, we observed an association and an excess of wild type alleles in non affected offspring for SNPs +61450 C>A, and +83897 T>A (Z=−2.17 p=0.03 and Z=−2.09 p=0.03 respectively) and a trend towards association and excess of G allele for SNP −243 A>G in affected offspring (Z=1.87, p=0.06). For haplotype analysis of SNPs +61450 C>A and +83897 T>A, the global test for association with obesity was significant (χ²=7.637, p-value=0.02). The protective wild type (C-T) haplotype was in excess in non affected offspring (Z=−1.94, p-value=0.05) (table 1). A significant association with the evidence of linkage was observed for SNP+61450 C>A. 17 nuclear families, with concordant affected sibpairs for (A-A) genotype, displayed a lod-score of 4.13, p=0.02 (as this lod-score was reached 20 times out of 1000 simulations, assessing a C allele frequency of 0.72, value in the control cohorts). When considering a C allele frequency of 0.68 (value in the morbidly obese cohorts) a p value of 0.06 was observed. Remaining sibpairs non-concordant for this genotype, displayed a lod score of 1.75. Thus, it is impossible to exclude that SNP+61450 C>A explains the observed linkage.

### Example 5

### Modulation of Insulin Secretion by GAD2 SNPs

As GABA release from β cells was suggested to strongly inhibit insulin secretion (Shi et al., 2000), we looked in non diabetic control subjects for a potential effect of GAD2 SNPs on fasting insulin and on the □□ cell function (HOMA-B %) assessed with the homeostasis model. We found that subjects homozygous (AA) for +83897 T>A showed lower fasting insulin levels and HOMA B indexes (pvalues for Kruskal-Wallis test: 0.0091 and 0.01 respectively for fasting insulin and HOMA B) (FIG. 6) suggesting a deleterious effect of GAD2 SNPs on insulin secretion, probably through an increase of the GABA pool in pancreatic β cells mediated by GAD65 higher enzymatic activity.

1. A method for diagnosing a predisposition for obesity, and in particular morbid obesity, in a human subject which comprises determining whether there is a germline alteration in the sequence of the 5′ flanking region of the gad2 gene, the coding sequence of which is represented by SEQ ID No 1, wherein said alteration is the presence of at least one of the following mutations: −243 A>G at nucleotide 2137 of SEQ ID No 2, −1.6 kb G>A at nucleotide 780 of SEQ ID No 2, −2004 A>T at nucleotide 376 of SEQ ID No 2, said alteration being indicative of a predisposition to obesity.

2. The method of claim 1, wherein said obesity is morbid obesity.

3. A method for diagnosing a predisposition for obesity in a human subject, from a sample from said subject, wherein the level of an expression product of the gad2 gene in said sample is investigated.

4. The method of claim 3, wherein said expression product is RNA or protein, or GABA.

5. The method of one of claim 1 to 4 further comprising a step consisting of detecting a protective haplotype for morbid obesity including alleles of SNP +61450 C>A, and +83897 T>A as depicted in SEQ ID No 16 and 17 respectively.

6. A primer or probe for detecting a predisposition for obesity selected from SEQ ID No 4 to 15.

7. A kit for detecting a predisposition for obesity comprising a set of primers or probes consisting of SEQ ID No 4, 5, 8, 9, 12 and 13 or a set of primers or probes consisting of SEQ ID No 6, 7, 10, 11, 14, and 15.

8. The kit according to claim 7 further comprising a primer or probe allowing detection of a protective haplotype consisting of 10 to 30 consecutive nucleotides of SEQ ID No 16 or 17 or of a sequence complementary thereof.

9. A method for screening potential obesity drugs which comprises: combining (i) a compound suspected of being an obesity drug, (ii) a GAD2 polypeptide and determining the amount of binding of the GAD2 polypeptide to said compound.

10. A method for screening potential obesity therapeutics which comprises:
combining (i) a GAD2 binding partner, (ii) a GAD2 polypeptide and (iii) a compound suspected of being a obesity therapeutic and determining the amount of binding of the GAD2 polypeptide to its binding partner.

11. The method of claim 10, wherein said GAD2 binding partner is L-glutamic acid.

12. A method for screening potential obesity therapeutics which comprises: combining (i) a gad2 gene binding partner, (ii) a gad2 gene and (iii) a compound suspected of being a obesity therapeutic and determining the amount of binding of the gad2 gene to its binding partner.

13. The method of claim 12, wherein said gad2 gene binding partner is IK2 (Ikaros 2).

14. A pharmaceutical composition comprising a pharmaceutically acceptable excipient with a compound identified with the method according to any of claims 9 to 13.

15. Use of a compound identified with the method according to any of claims 9 to 13, or of a composition according to claim 14 for the preparation of a drug intended for treatment of obesity, in particular morbid obesity.

16. Use of an antisense molecule or SiRNA complementary to the gad2 mRNA for the preparation of a drug intended for treatment of obesity, in particular morbid obesity.

17. The use according to claim 15 or 16 for the modulation of insulin secretion.

18. Use of a sense molecule comprising a fragment of the 5′ flanking region of the gad2 gene, especially comprising the −243 A>G variant (at nucleotide 2137 of SEQ ID No 2), within said region, for the manufacture of a drug intended for the treatment of obesity.

19. A transgenic non-human mammal having integrated into its genome the nucleic acid sequence of gad2, or coding sequence thereof, operatively linked to regulatory elements, wherein expression of said sequence increases the level of the GAD2 protein and/or the GABA pool in said mammal relative to a non-transgenic mammal of the same species.

20. A transgenic non-human mammal whose genome comprises a disruption of the endogenous gad2 gene, wherein said disruption comprises the insertion of a selectable marker sequence, and wherein said disruption results in said non-human mammal exhibiting a defect in GABA level as compared to a wild-type non-human mammal.

21. The mammal of claim 19 or 20 which is a mouse.

22. Use of a mammal according to any of claims 19 to 21, as a model for studying obesity, or for testing potential anti-obesity drugs.


SEQUENCE LISTING (15 sequences)

SEQ ID NO: 1 - DNA, 1758 bp, Homo sapiens
  gad2 gene
atggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataa

SEQ ID NO: 2 - DNA, 2382 bp, Homo sapiens
  5′ flanking region of gad2 gene nucleotides
gaaaaaaaaatcagttaaattcaggtgttttaatccgtttcttctttgggggttttgtgtgatttaaacgcttgcttttaagaacctttatgttttcaaccactcatccatagtagaaaagttctgcaaccctagactgctggcttgaaggaaaacctttgcaggatttgatatggatttcaacaaagaaccagcctctgcgaggctggagagagctgcggagctgccatgcctgaagtgcagatggctgaaccacaagtctttaggtttccggagttgttattgtggtgacctagagtgtcagagccaggagagcaagaaagaggagccaaactgagccctgagttttcgaccacccgggctcccacagcctggwacagacttcacctagcacgctcagtgccagccttcggcaggacgctatcaacgcccgactggwttcytgctctcatcctggcgcctgggcccagttgccatagtgtggatcccatgactcctcagggaacccctggactcaggcacgcgagaagaagacagcgctttgtggagagaattgaccagggacagttatgctcgagcacacaggacttgggcctgtatgcgtccagcatgggccccaggatgtcccttctaagcgagggtcgaggggtgctcgcccagacgggatccccgggtctctgctttgttagcagctttggtggctggttcaggaggtcagagaaataaaacgacttgtgaacacaatggaaatgacaggcgctctggccaggcgcggggaargcagccgcctcgggaagccgacctcagccctttcctctctctccctccctccgtctcccccagagccccggagctccgaggtgcacttgaagttcatctccactgccaggagaacgcagcgcaaactgtcaaagggctcccaatccttagggcgtcctccctgtctgcaatagctttttgtagaaaggaaatcatcagaamgattctgactttctcctcctttctttattagaaaaagagaaaccgcctcctaatacgcctcagagagaaccaatctcgcgcttccgggtcacccgctaccgcgaagattctctgggggcgagggggggcattggtttgaagccccttaaaacgagggccctgcaggcgatgccttctttcctactcggatttgtaaagccgagattgcttagttggaaaccctgttctcccctcccaggcgcacacagatcccccttacacgcaagcagcgggcgcttccacgcctccgcgggccaaggtcaccaaatgccctgattccatcccccacccgccatcaatcctgccgactctggccgctctgcctcattctcttccaagaagtttccattcgttttatttttttttccccagcccgaggtcctcagtagactccagcgtggattttaattgcctcaatcagcagtcattctccccagccgtcactcagagcctggacggtgggtcccgcgatctagcccttggctacgcaggaacggtgcgcccccgggtacggcgcggttcagcaggcaggcgtcaggttctaccaaggcgctgaaatgagcccatcagcgggtaggagcccttcccccgccgtcccctccccaggctcgtgaacggcgcctgatgcccgcccggggcgcgagctctcgaggtcgcagtgacctcagcacctgcttggaggaaaacggcgcgggaaccccgcttccttcccctcagctggagccagacctcaaacaaaaccccaatcgatgcacacagaaaactcctctgggccacgcttcccgcctcgccgaggtctccccagtctgcccctcgctgacgctggcgcgcagcsgctgtggcagcacccgggacagcggccgcccgcacttcccgcctctggctcgcccgaggacgcgctggcacgcctcccaccccctcactctgactccagctggcgtgcatggtctgcctcgcatcctcacgactcagctccctccctctctcgtgtttttttcctccgccgccccctcattcatccccactgggctccctttccctcaaatgctctggggctctccgcgctttcctgagtccgggctccgaggacccttaggtagtcccggtctcttttaargctccccggcttccaaagggttgccacgtccctaaaccctgtctccagctcgcatacacacacgcacagacacgcacgttttctgttcctgcgtgacacccgccctcgccgctcggccccgccggtccccgcgcggtgccctcctcccgccacacgggcacgcacgcgcgcgcagggccaagcccgaggcagctcgcccgcagctcgcactcgcaggcgacctgctccagtctccaaagccgatg

SEQ ID NO: 3 - protein, 519 aa, Homo sapiens
  DNA-binding protein Ikaros (Lymphoid transcription factor LyF-1)
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 4 - DNA, 20 bp, Artificial Sequence
  Chemically synthesized Primer to amplify SNP - 243
cctcaaatgctctggggctc

SEQ ID NO: 5 - DNA, 20 bp, Artificial Sequence
  Chemically synthesized Primer to amplify SNP - 243
ggtgtcacgcaggaacagaa

SEQ ID NO: 6 - DNA, 23 bp, Artificial Sequence
  Chemically synthesized Primer to amplify SNP - 243
gtctcttttaaagctccccggct

SEQ ID NO: 7 - DNA, 28 bp, Artificial Sequence
  Chemically synthesized Primer to amplify SNP - 243
cgggctccgaggacccttaggtagtccc

SEQ ID NO: 8 - DNA, 17 bp, Artificial Sequence
  Chemically synthesized Primer to amplify SNP - 1.6 kb
ctgaggcgtattaggag

SEQ ID NO: 9 - DNA, 17 bp, Artificial Sequence
  Chemically synthesized Primer to amplify SNP - 1.6 kb
ctcctaatacgcctcag

SEQ ID NO: 10 - DNA, 16 bp, Artificial Sequence
  Chemically synthesized Primer to amplify SNP - 1.6 kb
ggaaagcagccgcctc

SEQ ID NO: 11 - DNA, 30 bp, Artificial Sequence
  Chemically synthesized Primer to amplify SNP - 1.6 kb
tggaaatgacaggcgctctggccaggcgcg

SEQ ID NO: 12 - DNA, 21 bp, Artificial Sequence
  Chemically synthesized Primer to amplify SNP - 2004
tgttttcaaccactcatccat

SEQ ID NO: 13 - DNA, 17 bp, Artificial Sequence
  Chemically synthesized Primer to amplify SNP - 2004
agggacagttatgctcg

SEQ ID NO: 14 - DNA, 18 bp, Artificial Sequence
  Chemically synthesized Primer to amplify SNP - 2004
acagcctggtacagactt

SEQ ID NO: 15 - DNA, 23 bp, Artificial Sequence
  Chemically synthesized Primer to amplify SNP - 2004
tgagttttcgaccacccgggctc